(19) United States
(12) Patent Application Publication
(10) Pub. No.: US 2006/0241520 A1
(43) Pub. Date: Oct. 26, 2006

(54) System for prevention of work injuries

(76) Inventor: Hans Robertson, Jonkopong (SE)

Correspondence Address: HARNESS, DICKEY & PIERCE, P.L.C., P.O. BOX 8910, RESTON, VA, 20195, US

(21) Appl. No.: 10/544,612

(22) PCT Filed: Feb. 5, 2004
(86) PCT No.: PCT/SE04/00158

§ 371 Date: May 10, 2006

(30) Foreign Application Priority Data
Feb. 6, 2003 (SE) 0300338-1

(51) Int. Cl. A61B 5/103; A61B 5/00

(52) U.S. Cl. 600/595; 600/300; 600/587

(57) ABSTRACT

A system is disclosed for counteracting work-related stress injuries in an individual person when working in connection with at least one work-related object which permits the adjustment or use of different working positions, the object being provided with at least device for detecting its current adjustment or working position and delivering a signal containing information on this to a computer. The information is recorded and stored together with information on the current time. The computer also includes a device for recording and storing information relating to pain symptoms experienced by the individual together with information on the current time. The system perferably also includes a graphic dialogue box connected to the computer for communication with the individual and in which the individual marks the position and intensity of pain symptoms experienced, following which this information is transferred to the computer.

The invention relates to a system for alleviating and preventing work injuries, primarily in an office environment but also, for example, in forestry work and timber handling, building and construction work and other work situations.

### BACKGROUND OF THE INVENTION

In office environments where there is prolonged use of computers, work-related stress injuries often occur. These lead, among other things, to pains in the elbows, shoulders, neck and back etc.

In order to alleviate these, increasing use is now being made of furniture that will allow flexible working positions. This furniture can help to reduce the stresses or make them more variable.

This involves, among other things, the use of height-adjustable desks and chairs with a wider range of possible adjustments.

The pains experienced, however, are often of an individual nature and difficult to analyse. For this reason, the flexibility of the furniture is seldom utilised to good effect. Computer work can be absorbing and focussed and before one realises it one has been sitting in a wrong position for too long doing persistent harm.

It would therefore be desirable to supplement the adjustable furniture with a system that allows the individual to make effective use of its flexibility.

Similar problems and injuries can arise in sawing work when handling power saws, in building work when using working tools such as grinding and shaping machines and in other situations.

### SUMMARY OF THE INVENTION

The object of the present invention is to provide a system which is designed to map how an individual's work-related pain symptoms vary as a function of work activities and to make use of working positions, particularly on adjustable furniture. The system warns the individual when a situation is about to arise for which the mapping has shown that pain symptoms generally occur and it is therefore advisable to change working positions in order to prevent this.

The system comprises sensors on work equipment, particularly the items of adjustable furniture, which detect settings and adjustments of the tool or the relevant furniture, and sensors that detect computer-related activity such as keyboard and pointing device activities. The system also comprises the necessary hardware and software needed to record and store the status of the sensors as a function of the time and in appropriate cases also to record the nature of any pain symptoms experienced by the individual.

As data is thus gathered on the individual's behaviour and the resulting pain sensations, an analytical program (constructed as a neural network, for example), which is linked to the recording can identify correlations between the behaviour and pain symptoms. The individual can there be alerted and encouraged to alter his or her working position before it is too late. If such a warning and subsequent adjustment of the working position proves successful, the neural network can then further improve its preventive warning capability.

The system according to the invention has the characterising features specified in claim 1. Embodiments of the invention have the characterising features revealed by the other patent claims.

### DESCRIPTION

The invention will now be described on the basis of the following non-limitative examples of embodiment.

It is assumed that an individual has a work station comprising a height-adjustable desk, a computer with keyboard, display screen and pointing device, and also an office chair with adjustment facilities.

It is furthermore assumed that the height-adjustable desk is provided with a sensor which is capable of recording the height of the desk, and that a program on the computer records the keyboard activity and point device movements and what computer program the individual is working with.

The chair is also provided with sensors which record chair movements, the adjustment position of the chair and where appropriate also the seated posture of the individual. The latter can be accomplished, for example, by one or more load cells fitted to the chair. In a preferred embodiment of the invention the system also comprises means, sensors, located on the individual for recording stresses and/or movements and for transmitting this information and storing and processing it on the computer.

The sensors are appropriately equipped for wireless communication and provided with long-life battery and communicate sensor data to hardware and software which are suited to the purpose and linked to the computer.

On the computer there is also interactive software allowing the individual to record pain symptoms. This dialogue can either be activated by the individual himself when he feels pain or automatically activated when the program registers that the individual is changing or intends to change his working position. Communication with the individual can be achieved by means of a graphic dialog box in which the individual indicates his position and the intensity of pain symptoms experienced, following which this information is transferred to the computer. For example, should the individual activate a desk height adjustment, a dialog box can appear on the computer display screen, which initially asks whether there has been any change in the pain experienced by the individual and then shows the latest recorded entry. A suitable design variant may be to display a body image with predefined areas where the individual usually feels pain. The individual can than mark where the pain is felt and the intensity of the pain sensation. Where appropriate the individual can also append explanatory comments which can then be used in consultation with an occupational therapist.

The program can then either itself suggest a change in working position and if the system is designed to control furniture adjusting devices the adjustment can be made automatically, otherwise the individual himself is allowed to control the adjustment which is then registered by the relevant sensors.

As time goes on the computer gathers sensor data and pain records. The computer program is assumed to contain a self-learning analytical part, in the form of a neural network, for example. When sufficient data has been analysed, the program can begin to give warning of situations which the neural network, from experience, has found to be generally conducive to pain sensations. In the event of such warnings the individual can again be questioned about his pain condition, which gives the neural network the opportunity to further refine its warnings.

The computer preferably also comprises means of processing stored information and suggesting changes to any parameter on the basis of the processed information.

In a further refinement a window on the computer screen may be used to display a historical picture of sensor data and pain information over time, and a signal emitted which gradually becomes stronger and reminds the individual that it is now becoming more urgent to change working position.

1. System for counteracting work-related stress injuries in an individual person when working in connection with at least one work-related object which permits the adjustment or use of different working positions, the object being provided with at least one means of detecting its current adjustment or working position and delivering a signal containing information on this to a computer, in which the information is recorded and stored together with information on the current time, the computer also comprising means of recording and storing information relating to pain symptoms experienced by the individual together with information on the current time, and an analytical program linked to the recording comprises means of collating and processing information in order to identify such combinations of sensor signals and other information which have been shown to lead to pain sensations in the individual.

2. System according to claim 1, further comprising a graphic dialogue box connected to the computer in which the individual marks the position and intensity of pain symptoms experienced, following which this information is transferred to the computer.

3. System according to claim 1, wherein the object is an item of furniture.

4. System according to claim 1, wherein the object is a working tool.

5. System according to claim 1, further comprising means located on the individual for recording stresses and/or movement and for transferring this information, and storing and processing it on the computer.

6. System according to claim 1, wherein at least one working tool used by the individual is provided with means of recording how the working tool is used and for transferring this information and storing and processing it on the computer.

7. System according to claim 1, wherein the computer comprises means of requesting the input of information from the individual when a given set parameter is modified.

8. System according to claim 1, wherein the computer also comprises means of processing stored information and suggesting changes to any parameter.

9. System according to claim 1, wherein the working tool is a computer with associated means of inputting and reading out information.

10. System according to claim 9, further comprising means of detecting activity for inputting information.

11. System according to claim 9, further comprising means of registering which program is being used on the computer that constitutes the working tool.

12. System according to claim 9, wherein one and the same computer is both working tool and computer for receiving, storing, processing and outputting system information.

13. System according to claim 1, wherein the system delivers warning information as soon as it identifies such combinations of sensor signals and other information that have been shown to lead to pain sensations in the individual.

14. System according to claim 13, further comprising means of suggesting changes to working conditions.

15. System according to claim 1, further comprising means of displaying selected parts of sensor signals, pain indications and other information to the individual as a function of the time.

16. System according to claim 1, further comprising means of controlling adjusting meechanisms of the working tool(s).

17. System according to claim 2, wherein the object is an item of furniture.

18. System according to claim 10, further comprising means of registering which program is being used on the computer that constitutes the working tool.
